(19) United States
(12) Patent Application Publication
(10) Pub. No.: US 2025/0035507 A1
(43) Pub. Date: Jan. 30, 2025

(54) SOLID-PROPELLANT THRUSTER WITH AN INTEGRATED CONTROL MEANS AND SYSTEM FOR CONTROLLING AGEING OF THE THRUSTER

(71) Applicant: ROXEL FRANCE, Saint-Médard-en-Jalles (FR)

(72) Inventor: Claude ALARY, Saint-Médard-en-Jalles (FR)

(21) Appl. No.: 18/785,576

(22) Filed: Jul. 26, 2024

(30) Foreign Application Priority Data
Jul. 27, 2023 (FR) 2308136

(51) Int. Cl. G01M 5/00; F02K 9/10; F02K 9/34

(52) U.S. Cl. CPC G01M 5/0066; F02K 9/10; F02K 9/34; G01M 5/0033

(57) ABSTRACT

A solid-propellant thruster comprises a casing accommodating a solid-propellant charge, an axial channel being formed in the solid-propellant charge and enabling combustion of the solid-propellant charge. The casing carries a solid-propellant specimen arranged in the channel and a transducer arranged outside the casing and opposite the specimen so as to enable the emission of a vibratory impulse throughout the specimen and to transmit a response thereof.

This application claims priority to French Patent Application 23 08136 filed Jul. 27, 2023, the entire disclosure of which is incorporated by reference herein.

The invention relates to a solid-propellant thruster with an integrated control means and a system for controlling ageing of the thruster.

Many rocket-type ballistic projectiles implement the combustion of a solid-propellant to deliver a thrust force ensuring movement thereof.

After manufacture, ballistic projectiles may be stored over periods sometimes very long which could reach several years. It is essential to know the condition of the solid-propellant before use thereof in order to verify whether it is capable of fulfilling its function, to assess the safety level inherent to operation thereof.

It is possible to define the apparent age of the solid-propellant corresponding to an ageing level that is actually noticed. This allows assessing the service life that is still available for the ballistic projectile implementing the solid-propellant.

Solid-propellants contain both a fuel and an oxidiser and the knowledge of some mechanical characteristics of the solid-propellant, like the Young's modulus or the shear modulus, allow estimating its apparent age. The mechanical characteristics may be derived from the response of the material to the impulse of a vibratory wave.

It is known, in particular from the document FR 2 954 498 A1, to implement a testing device containing, within a case, a solid-propellant sample and a vibratory system. The testing device may be placed proximate to the ballistic projectile and even inside the latter. In an attempt to obtain a comparable ageing for the ballistic projectile and for the testing device.

However, the testing device described in the document FR 2 954 498 A1 is not suitable for operation on an industrial scale. Several drawbacks have been identified by the Applicant:
The device is heavy and bulky inside the thruster.
The surrounding conditions of the solid-propellant sample of the testing device are not the same as those of the solid-propellant of the thruster because of the sample is contained inside the case of the testing device. In particular, the oxidation of the sample does not occur at the same rate as that of the solid-propellant of the thruster.
The set-up of the testing device inside the thruster results in the presence of an electrical energy source inside the thruster. This poses a danger of ignition of the thruster during a sample testing phase.
The presence of components other than solid-propellant inside the thruster could disturb operation thereof.

The invention aims to address all or part of the aforementioned problems by providing a solid-propellant thruster equipped with a testing specimen allowing emitting a vibratory impulse, from outside the thruster, throughout the specimen and transmitting a response thereof.

To this end, an object of the invention is a solid-propellant thruster comprising a casing accommodating a solid-propellant charge, an axial channel being formed in the solid-propellant charge and enabling combustion of the solid-propellant charge, the casing carrying a solid-propellant specimen arranged in the channel and a transducer arranged outside the casing and opposite the specimen so as to enable the emission of a vibratory impulse throughout the specimen and to transmit a response thereof.

Advantageously, the channel extends between two axial ends of the thruster, the casing having an orifice for the passage of the specimen communicating with the channel at one of its ends.

Advantageously, the thruster further includes a disk fastened to the casing at the orifice and having an inner face directed towards the channel and on which the specimen is fastened as well as an outer face directed opposite the channel and on which the transducer is fastened.

Advantageously, the casing has a cylindrical recess in which the disk is accommodated, the orifice for the passage of the specimen being formed in the bottom of the cylindrical recess.

Advantageously, the specimen is fastened on an inner face of the disk by means of a glue.

The solid-propellant charge may comprise a polymer binder and the glue is advantageously based on the same polymer binder.

Advantageously, the solid-propellant of the specimen is identical to that one of the charge.

Advantageously, the transducer comprises electrical connections allowing receiving a first signal configured so that the transducer emits the vibratory impulse and allowing recovering a second signal representative of the response of the specimen to the vibratory impulse.

Another object of the invention is a system for controlling ageing of a thruster according to the invention, comprising an electronic module configured to:
temporarily connect to the transducer,
emit a first signal towards the transducer enabling it to form the vibratory impulse,
receive a second response signal from the transducer,
deduce from the received signal a parameter representative of an ageing condition of the charge of the thruster.

The invention will be better understood and other advantages will appear upon reading the detailed description of one embodiment given as example, which description is illustrated by the appended drawings wherein:

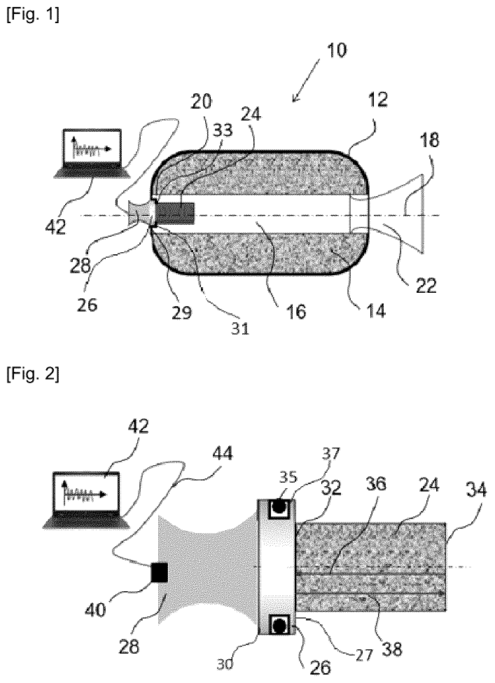

FIG. 1 schematically shows a solid-propellant thruster according to the invention;

FIG. 2 shows in more detail components allowing testing the solid-propellant of the thruster of FIG. 1.

For clarity, the same elements bear the same references in the different figures.

FIGS. 1 and 2 schematically show a thruster 10 implemented, for example, in a ballistic projectile. The thruster 10 comprises a solid-propellant charge 14 inside a casing 12. The solid-propellant contains both a fuel and an oxidiser. For example, the solid-propellant is a composite solid-propellant based on a polyurethane binder as described in the French patent FR 3 108 331 B3 filed on the name of the Applicant. The invention is not limited to this solid-propellant type and any solid-propellant type may be implemented in the context of the invention.

The combustion of the solid-propellant ensures thrust of the thruster and the casing 12 should withstand the pressure and the temperature due to the combustion. To withstand the combustion temperature, it is possible to integrate a heat shield between the casing 12 and the solid-propellant charge. To withstand pressure, different materials may be implemented like, for example, a metal material such as a steel or a carbon fibre based material. Other materials may be implemented and other constraints than pressure and temperature could appear later on for the implementation of the invention.

A longitudinal channel 16, extending according to an axis 18, is formed in the solid-propellant charge 14. The channel 16 extends between two axial ends 20 and 22 of the thruster 10. One of the ends 20 of the channel 16 is herein plugged by a disk 26 and the other end 22 of the channel 16 forms a nozzle through which the solid-propellant combustion products ensuring propulsion are ejected.

According to the invention, the thruster 10 comprises a solid-propellant specimen 24 arranged in the channel 16 and herein fastened on an inner face 27 of the disk 26. Advantageously, the solid-propellant specimen 24 is made of the same solid-propellant as that one of the solid-propellant charge 14. Alternatively, it is possible to implement for the specimen 24 a solid-propellant other than that one of the charge 14 but having close physical properties, in particular in terms of ageing.

The thruster 10 further comprises a transducer 28 fastened on an outer face 30 of the disk 26 and arranged opposite the specimen 24 so as to enable the emission of a vibratory impulse throughout the specimen 24 and to transmit a response thereof. The channel 16 may be formed directly in the solid-propellant charge 14. In this case, the specimen 24 is advantageously bare. More specifically, the solid-propellant of the specimen 24 as well as that one of the charge 14 are directly exposed to air within the channel 16. Thus, the solid-propellant of the charge 14 and that one of the specimen 24 react in the same way to the environment of the channel 16. More specifically, during storage of the thruster 10, temperature and humidity variations may occur. These two parameters are critical in ageing of the solid-propellant. Other parameters may also intervene like for example pressure but are, a priori, less critical. However, exposing the solid-propellant of the charge 14 and that one of the specimen 24 to the same environment allows taking account of any environment parameter type. This identical exposure also allows adapting to different solid-propellant compositions that could age differently depending on the environment; while keeping the same material for the solid-propellant of the charge 14 and that one of the specimen 24, these will age in a similar way as they are exposed to the same environment conditions.

FIG. 2 shows the transducer 28, the disk 26 and the specimen 24 in more detail. The transducer 28 is arranged over the outer face 30 of the disk 26 carrying the specimen 24 over its inner face 27, herein parallel to the outer face 30, and is configured to emit a vibratory impulse in the specimen 24. For example, the vibratory impulse is obtained by means of an ultrasonic acoustic-type wave emitted by the transducer 28 and crossing the disk 26 and the specimen 24. In particular, a disk 26 made of steel has good properties of transmission of an ultrasonic-type vibratory impulse. More generally, the material of the insert that the disk 26 forms is selected so that it could transmit a mechanical impulse while being barely subjected to the risk of ageing like the solid-propellant.

In the example shown in FIGS. 1 and 2, the specimen 24 has two parallel faces, one 32 in contact with the inner face 27 of the disk 26 and the other one 34 clear and exposed to air present in the channel 16. The vibratory impulse emitted by the transducer 28 crosses the specimen 24 once from its face 32 up to its face 34, is reflected at least partially on the face 34, crosses the disk 26 again before being received by the transducer 28. The pathway of the vibratory impulse, so-called forward pathway, from the face 32 towards the face 34 is materialised by an arrow 36 and the pathway of the vibratory impulse, so-called return pathway, from the face 34 towards the face 32 is materialised by an arrow 38. Other specimen 24 shapes are possible to implement the invention. The faces 32 and 34 may be non-parallel. The faces 32 and 34 are not necessarily planar. For example, the inner face 27 of the disk 26 over which the specimen 24 is arranged may be non-planar and the face 32 of the specimen 24 advantageously conforms to the shape of the inner face 27 in order to promote the transmission of the vibratory impulse directly from the disk 26 towards the specimen 24 without crossing any air gap that might be found between the inner face 27 of the disk 12 and the face 32 of the specimen 24.

The thickness of the disk 12 enables it to withstand the temperature/pressure prevailing in the combustion chamber but, where necessary, a heat-shielding sheet may be interposed between these two faces 27 and 32 to thermally protect the disk 26, without this fundamentally disturbing the acoustic principle of the system.

Advantageously, the specimen 24 is glued over the inner face 27 of the disk 26 whereas the transducer 28 is glued over the outer face 30 of the disk 26. Thus, no mechanical component likely to disturb the vibratory impulse is present in the fixtures of the specimen 24 and of the transducer 28. Gluing also allows limiting the formation of an air gap between the inner face 27 of the disk 26 and the face 32 of the specimen 24 by completely spreading between the faces 27 and 32. The same applies on the transducer 28 side. The solid-propellant may comprise a polymer binder, as described for example in the patent FR 3 108 331 B3. It is then advantageous to implement a glue 39 based on the same binder as that one implemented in the solid-propellant to fasten the specimen 24 over the inner face 27 of the disk 26, or the transducer 28 over its opposite face 30. In particular, this glue type allows less disturbing the propagation of the vibratory impulse at the interface between the glue and the solid-propellant.

Alternatively, gluing the specimen 24 directly onto the inner face of the casing 12 may be considered with a casing plugging the end 20 of the channel 16.

Fastening the disk 26 to the casing may be carried out in different ways. It is then accommodated in a cylindrical recess 29 of the casing 12 and fastened to the latter by screwing (not shown in the figures for simplicity). The bottom 31 of the recess 29 has an orifice 33 to let the specimen 24 pass. For example, alternatively, it is also possible to mechanically clamp the disk 26 on the outer face of the casing 12.

Moreover, a sealing gasket 35 is interposed between the disk 26 and the casing 12, while being herein accommodated in a groove 37 formed in the disk 26.

In FIGS. 1 and 2, the transducer 28 is shown as a one-piece component configured to emit a vibratory impulse and to receive a response to the emitted vibratory impulse. In practice, it is possible to distinguish the two functions: emission and reception of the vibratory impulse, a first component to emit the impulse and a second one to receive the vibratory impulse reflected by the face 34 after having crossed the specimen 24. The transducer 28 may generate the vibratory impulse by means of a piezoelectric ceramic. This ceramic type also allows receiving the reflected wave, forming the response to the vibratory impulse, and outputting a corresponding electrical signal.

In the illustrated embodiment, the transducer 28 comprises electrical connections 40 allowing temporarily connecting the transducer 28. The electrical connections 40 and the transducer 28 are outside the casing 12, which allows avoiding any electrical signal penetrating into the casing to make the transducer 28 function. Thus, only the vibratory impulse penetrates into the casing 12. The electrical connections 40 allow connecting an electronic module 42 herein represented in the form of a personal computer. The electronic module 42 may be connected to the transducer 28 by means of a cable 44. Alternatively, the electronic module 42 may be connected to the transducer 28 by means of a wireless link.

The electronic module 42 is configured to generate and emit a signal towards the transducer 28 enabling it to form the vibratory impulse. The electronic module 42 is also configured to receive a response signal from the transducer 28, which signal corresponds to the vibratory response of the specimen 24.

The electronic module 42 is configured to analyse the response signal of the transducer 28 and to deduce therefrom a parameter representative of the ageing condition of the solid-propellant of the specimen 24 and therefore of the ageing condition of the solid-propellant of the charge 14. For example, this deduction is done by comparing the received signal with memorised signals representative of different ageing conditions. It is also possible to determine some mechanical parameters of the solid-propellant like its Young's modulus or its shear modulus from the vibratory response. The evolutions of these mechanical parameters reflect the ageing condition of the solid-propellant.

In this respect, it should be noted that the arrangements according to the invention allow determining the residual service life of a thruster in particular without having to know the conditions in which the thruster has been stored to determine the ageing state of the solid-propellant.

1. A solid-propellant thruster comprising a casing accommodating a solid-propellant charge, an axial channel being formed in the solid-propellant charge and enabling combustion of the solid-propellant charge, the casing carrying a solid-propellant specimen arranged in the channel and a transducer arranged outside the casing and opposite the specimen so as to enable the emission of a vibratory impulse throughout the specimen and to transmit a response thereof.

2. The thruster according to claim 1, wherein the channel extends between two axial ends of the thruster, the casing having an orifice for the passage of the specimen communicating with the channel at one of its ends.

3. The thruster according to claim 2, further including a disk fastened to the casing at the orifice and having an inner face directed towards the channel and on which the specimen is fastened as well as an outer face directed opposite the channel and on which the transducer is fastened.

4. The thruster according to claim 3, wherein the casing has a cylindrical recess in which the disk is accommodated, the orifice for the passage of the specimen being formed in the bottom of the cylindrical recess.

5. The thruster according to claim 3, wherein the specimen is fastened on an inner face of the disk by means of a glue.

6. The thruster according to claim 5, wherein the solid-propellant charge comprises a polymer binder and wherein the glue is based on the same polymer binder.

7. The thruster according to claim 1, wherein the solid-propellant of the specimen is identical to that one of the charge.

8. The thruster according to claim 1, wherein the transducer comprises electrical connections allowing receiving a first signal configured so that the transducer emits the vibratory impulse and allowing recovering a second signal representative of the response of the specimen to the vibratory impulse.

9. A system for controlling ageing of a thruster according to claim 1, comprising an electronic module configured to:
temporarily connect to the transducer,
emit a first signal towards the transducer enabling it to form the vibratory impulse,
receive a second response signal from the transducer,
deduce from the received signal a parameter representative of an ageing condition of the charge of the thruster.
